(19) United States
(12) Patent Application Publication
(10) Pub. No.: US 2009/0005407 A1
(43) Pub. Date: Jan. 1, 2009

(54) PARTICULATE (3'-CHLOROBIPHENYL-4-YL)(1-(PYRIMIDIN-2-YL)PIPERIDIN-4-YL)METHANONE AND METHODS OF ITS USE

(76) Inventor: Goutam Muhuri, Belle Mead, NJ (US)

Correspondence Address: LEXICON PHARMACEUTICALS, INC., 8800 TECHNOLOGY FOREST PLACE, THE WOODLANDS, TX, 77381-1160, US

(21) Appl. No.: 12/147,589

(22) Filed: Jun. 27, 2008

Related U.S. Application Data
(60) Provisional application No. 60/946,765, filed on Jun. 28, 2007.

(51) Int. Cl. C07D 401/04; A61K 31/506; A61P 25/28; A61P 25/18

(52) U.S. Cl. 514/275; 544/297

(57) ABSTRACT

Particulate (3′-chlorobiphenyl-4-yl)(1-(pyrimidin-2-yl)piperidin-4-yl)methanone is disclosed. Also disclosed are pharmaceutical formulations and dosage forms comprising particulate (3′-chlorobiphenyl-4-yl)(1-(pyrimidin-2-yl)piperidin-4-yl) and methods of their use.

This application claims priority to U.S. provisional application No. 60/946,765, filed Jun. 28, 2008, the entirety of which is incorporated herein by reference.

### 1. FIELD OF THE INVENTION

This invention relates to particulate (3′-chlorobiphenyl-4-yl)(1-(pyrimidin-2-yl)piperidin-4-yl)methanone, compositions comprising it, and methods of its use.

### 2. BACKGROUND OF THE INVENTION

Different solid forms of the same compound can have substantially different properties. For example, the amorphous form of a drug may exhibit different dissolution characteristics and bioavailability patterns than its crystalline form(s), properties which can affect how the drug must be administered to achieve optimal effect. Amorphous and crystalline forms of a drug may also have different handling properties (e.g., flowability, compressibility), dissolution rates, solubilities and stabilities, all of which can affect the manufacture of dosage forms. Similarly, the mean particle size and shape of a compound can affect its dissolution and solubility properties. See, e.g., U.S. Pat. No. 5,145,684.

The compound (3′-chlorobiphenyl-4-yl)(1-(pyrimidin-2-yl)piperidin-4-yl)methanone is an inhibitor of the Na⁺-dependent proline transporter, and is believed to be useful in the treatment of cognitive diseases and disorders. See U.S. patent application Ser. Nos. 11/433,057 and 11/433,626, both filed May 12, 2006, and U.S. provisional 60/835,677, filed Aug. 4, 2006. Because the compound is poorly water soluble, forms with improved physical-chemical properties are desired in order to facilitate its delivery to patients.

### 3. SUMMARY OF THE INVENTION

This invention is directed, in part, to particulate (3′-chlorobiphenyl-4-yl)(1-(pyrimidin-2-yl)piperidin-4-yl)methanone.

One embodiment of the invention encompasses pharmaceutical compositions comprising particulate (3′-chlorobiphenyl-4-yl)(1-(pyrimidin-2-yl)piperidin-4-yl)methanone. Another embodiment encompasses methods of improving cognitive performance, and of treating, managing and/or preventing various diseases and disorders, using particulate (3′-chlorobiphenyl-4-yl)(1-(pyrimidin-2-yl)piperidin-4-yl)methanone.

### 4. BRIEF DESCRIPTION OF THE FIGURES

Certain aspects of the invention may be understood with reference to the attached figures.

FIG. 1 is a X-ray powder diffraction pattern of a crystalline solid form of (3′-chlorobiphenyl-4-yl)(1-(pyrimidin-2-yl)piperidin-4-yl)methanone. The spectrum was obtained using a Shimadzu XRD-6000 diffractometer configured as follows: X-ray tube [Cu (1.54060 Å), 40.0 kV, 40.0 mA]; scan range [3.00 to 45.0 degrees, 0.0400 degree step size]; count time [1.20 seq].

FIG. 2 is a FT-Raman spectrum of a crystalline solid form of (3′-chlorobiphenyl-4-yl)(1-(pyrimidin-2-yl)piperidin-4-yl)methanone. The spectrum was obtained using a Bruker RFS100 spectrometer: 1064 nm excitation (100 mW); 64 scans.

### 5. DETAILED DESCRIPTION OF THE INVENTION

This invention is directed, in part, to particulate (3′-chlorobiphenyl-4-yl)(1-(pyrimidin-2-yl)piperidin-4-yl)methanone, which is an inhibitor of the Na⁺-dependent proline transporter. When administered to mice, the compound has been shown to increase learning and memory. See U.S. patent application Ser. Nos. 11/433,057 and 11/433,626, both filed May 12, 2006.

This invention is also directed to dosage forms comprising particulate (3′-chlorobiphenyl-4-yl)(1-(pyrimidin-2-yl)piperidin-4-yl)methanone, and to methods of using particulate (3′-chlorobiphenyl-4-yl)(1-(pyrimidin-2-yl)piperidin-4-yl)methanone for the improvement of cognitive performance and for the treatment, prevention and/or management of diseases and disorders such as Alzheimer's disease, autism, cognitive disorders, dementia, learning disorders, and short- and long-term memory loss.

### 5.1. Definitions

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “mean particle size” has the same meaning as X₅₀.

Unless otherwise indicated, the term “particulate,” when used to describe a compound or composition, means that the compound or composition has an mean particle size of less than about 10 μm.

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or to prevent its recurrence. A prophylactically effective amount of a compound means an amount of therapeutic agent, alone or in combination with other agents, which provides a prophylactic benefit in the prevention of the disease or condition. The term “prophylactically effective amount” can encompass an amount that improves overall prophylaxis or enhances the prophylactic efficacy of another prophylactic agent.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder or one or more of its symptoms, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, the term “X₁₀” refers to the maximum particle size of the smallest ten percent, by volume, of particles within a particulate solid, as determined by the method described below in the Examples.

Unless otherwise indicated, the term “X₅₀” refers to the maximum particle size of the smallest fifty percent, by volume, of particles within a particulate solid, as determined by the method described below in the Examples.

Unless otherwise indicated, the term “X₉₀” refers to the maximum particle size of the smallest ninety percent, by volume, of particles within a particulate solid, as determined by the method described below in the Examples.

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

Unless otherwise indicated, the phrase “between X and Y” encompasses values between X and Y as well as X and Y themselves. Similarly, the phrases “between about X and about Y” and “between about X and Y” both refer to values between about X and about Y, including about X and about Y.

It should also be noted that any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. Structures that represent compounds with one or more chiral centers, but which do not indicate stereochemistry (e.g., with bolded or dashed lines), encompasses pure stereoisomers and mixtures (e.g., racemic mixtures) thereof. Similarly, names of compounds having one or more chiral centers that do not specify the stereochemistry of those centers encompass pure stereoisomers and mixtures thereof.

### 5.2. Particulate (3′-Chlorobiphenyl-4-yl)(1-(pyrimidin-2-yl)piperidin-4-yl)methanone

This invention is directed to particulate (3′-chlorobiphenyl-4-yl)(1-(pyrimidin-2-yl)piperidin-4-yl)methanone:

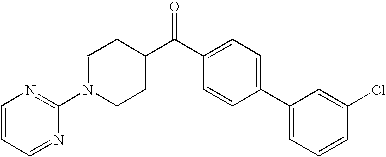

One embodiment of the invention is directed to particulate crystalline (3′-chlorobiphenyl-4-yl)(1-(pyrimidin-2-yl)piperidin-4-yl)methanone.

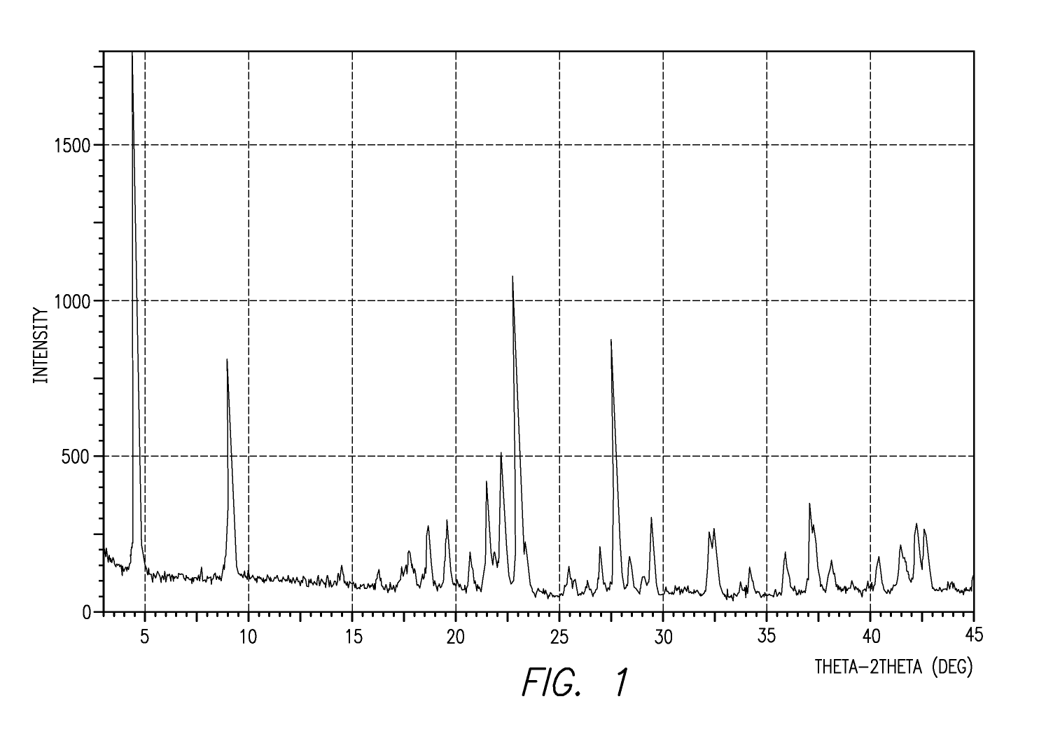

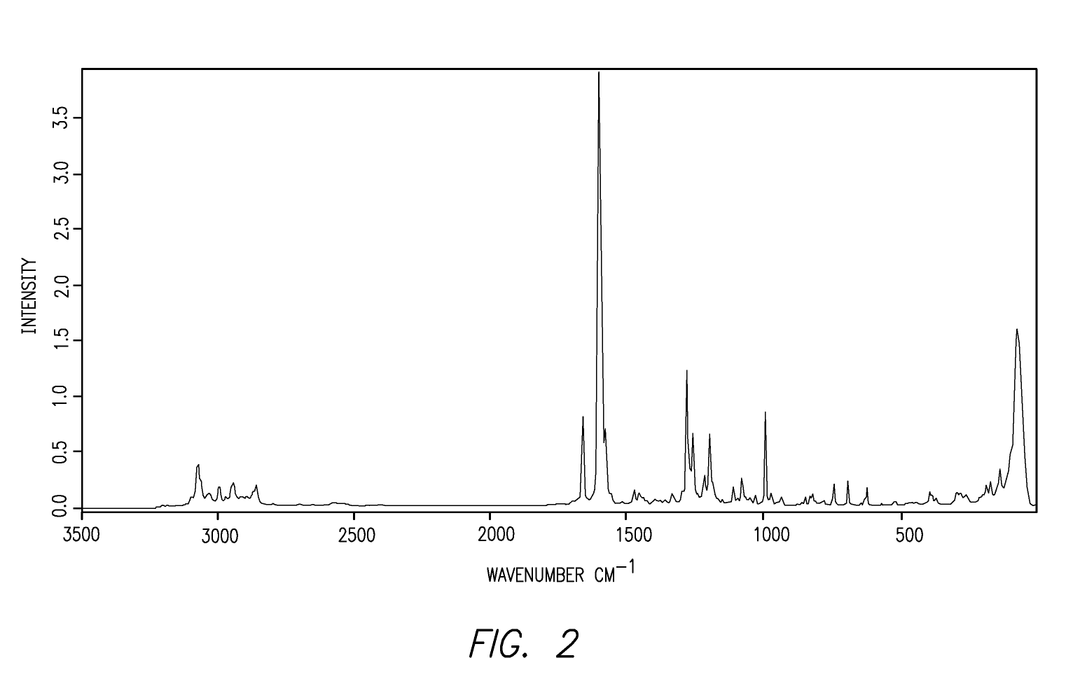

A particular crystalline form of (3′-chlorobiphenyl-4-yl)(1-(pyrimidin-2-yl)piperidin-4-yl)methanone has a melting point of about 117° C. (e.g., ±1.5° C.) as determined by DSC. The form provides a X-ray powder diffraction (XRPD) pattern that contains peaks at about 4.7, 9.3, 18.8, 19.7, 22.4, 23.2, 27.9, 29.6, 32.3, 32.6, 37.2, 41.5, 42.3, and/or 42.7 degrees 2θ. As those skilled in the art are well aware, the relative intensities of peaks in a XRPD pattern of a crystalline form can vary depending on how the sample is prepared and how the data is collected. With this in mind, an example of a XRPD pattern of this crystalline form is provided in FIG. 1. An example of an FT-Raman spectrum of this crystalline form is provided in FIG. 2.

Certain embodiments of this invention encompass (3′-chlorobiphenyl-4-yl)(1-(pyrimidin-2-yl)piperidin-4-yl)methanone having an mean particle size of less than about 10.0, 8.0, 6.0, 5.0, 4.0, 3.0, 2.0, 1.0, 0.75, 0.50, 0.25, or 0.15 μm. In particular embodiments, the mean particle size is less than about 1.0, 0.75, 0.50, 0.25, or 0.15 μm. In one embodiment, the mean particle size is between about 130 to about 170 nm. In another, it is between about 450 to about 550 nm.

Certain embodiments encompass (3′-chlorobiphenyl-4-yl)(1-(pyrimidin-2-yl)piperidin-4-yl)methanone having a X₁₀ of less than about 5, 4, 3, 2, or 1 μm.

Certain embodiments encompass (3′-chlorobiphenyl-4-yl)(1-(pyrimidin-2-yl)piperidin-4-yl)methanone having a X₉₀ of less than about 20, 15, 13, 11, 10, 9, or 8 μm.

### 5.3. Methods of Treatment

One embodiment of the invention encompasses a method of improving the cognitive performance of a human patient, which comprises administering to the patient an effective amount of a compound or composition of the invention. Examples of improved cognitive performance include enhanced learning (e.g., learning more quickly), improved comprehension, improved reasoning, and improved short- and/or long-term memory.

Another embodiment encompasses a method of treating, managing or preventing a cognitive disorder (e.g., difficulty in thinking, reasoning, or problem solving), memory loss (short- and long-term), or a learning disorder (e.g., dyslexia, dyscalculia, dysgraphia, dysphasia, dysnomia), which comprises administering to the patient an effective amount of a compound or composition of the invention.

Another embodiment encompasses a method of treating, managing or preventing a disease or disorder, or a cognitive impairment associated therewith, in a human patient, which comprises administering to the patient a therapeutically or prophylactically effective amount of a compound or composition of the invention. Examples of diseases and disorders include age-associated memory impairment, Alzheimer's disease, Attention-Deficit/Hyperactivity Disorder (ADD/ADHD), autism, Down syndrome, Fragile X syndrome, Huntington's disease, Parkinson's disease, and schizophrenia. Additional disorders include adverse sequelae of brain damage caused by, for example, oxygen starvation, traumatic injury, heart attack or stroke.

The invention also encompasses methods of treating, preventing and managing dementia, including dementia associated with metabolic-toxic, structural and/or infectious causes.

Metabolic-toxic causes of dementia include: anoxia; B₁₂ deficiency; chronic drug, alcohol or nutritional abuse; folic acid deficiency; hypercalcemia associated with hyperparathyroidism; hypoglycemia; hypothyroidism; organ system failure (e.g., hepatic, respiratory, or uremic encephalopathy); and pellagra.

Structural causes of dementia include: amyotrophic lateral sclerosis; brain trauma (e.g., chronic subdural hematoma, dementia pugilistica); brain tumors; cerebellar degeneration; communicating hydrocephalus; irradiation to frontal lobes; multiple sclerosis; normal-pressure hydrocephalus; Pick's disease; progressive multifocal leukoencephalopathy; progressive supranuclear palsy; surgery; vascular disease (e.g., multi-infarct dementia); and Wilson's disease.

Infectious causes of dementia include: bacterial endocarditis; Creutzfeldt-Jakob disease; Gerstmann-Sträussler-Scheinker disease; HIV-related disorders; neurosyphilis; tuberculous and fungal meningitis; and viral encephalitis.

The amount of compound administered to a patient will depend on the route of administration and on the condition being treated, managed or prevented, and can be readily determined by physicians. Example dosing regimens include 150, 600 and 1200 mg/day by oral administration.

### 5.4. Pharmaceutical Formulations

This invention encompasses pharmaceutical formulations (e.g., bulk drug formulations, dosage forms) comprising particulate (3′-chlorobiphenyl-4-yl)(1-(pyrimidin-2-yl)piperidin-4-yl)methanone, or pharmaceutically acceptable salts or solvates thereof. Preferred dosage forms are suitable for oral administration, and may be solid (e.g., tablets, capsules) or liquid (e.g., liquid suspensions). Suitable excipients and diluents are known to those of ordinary skill in the art. See, e.g., Remington's Pharmaceutical Sciences, 18^{th} ed., Mack Publishing, Easton Pa. (1990); Handbook of Pharmaceutical Excipients, 4^{th} ed., R. Rowe, et al., eds., Pharmaceutical Press (2003).

### 6. EXAMPLES

The compound (3′-chloro-biphenyl-4-yl)-(1-pyrimidin-2-yl-piperidin-4-yl)-methanone was prepared from an intermediate, (3′-chloro-biphenyl-4-yl)-piperidine-4-yl-methanone hydrochloride, as described below.

### 6.1. Preparation of (3′-Chloro-biphenyl-4-yl)-piperidine-4-yl-methanone Hydrochloride

(3′-Chloro-biphenyl-4-yl)-piperidine-4-yl-methanone hydrochloride was prepared by three different methods, identified below as A, B and C.

Method A:

3-Chlorophenyl boronic acid (Alfa Aesar, purity 97%)(40.7 g, 261.19 mmol, 1.4 eq) was dissolved in isopropanol (Aldrich, ACS reagent grade) (800 ml) under nitrogen atmosphere. This was added to a solution of aqueous potassium carbonate (77 g in 150 ml water), bis(triphenylphosphine)palladium(II) dichloride (PdCl₂(PPh₃)₂) (0.65 g, 0.93 mmol, 0.5 mol. eq.) and (4-bromophenyl)(piperidine-4-yl)methanone (50 g, 187 mmol, 1 eq) were stirred at 80° C. for three hours and deemed complete by LC/MS. After the reaction mixture cooled down to 50° C., it was filtered through celite pad, washed with methanol (1 liter). The filtrate was diluted with water (200 ml), then the organic solvent removed under reduced pressure. The resulting crude product was dissolved in ethyl acetate (800 ml) and washed with 1N sodium hydroxide (2×40 ml) and water (1×40 ml).

The organic layer was stirred with aqueous lactic acid (64 g of 85% lactic acid in 600 ml of water) at 50° C. for 20 minutes. After the organic layer was separated (solution assay indicated 8% of product present in the organic layer, which can be captured by additional lactic acid extraction), the aqueous layer was washed with ethyl acetate (2×100 ml). The aqueous layer was separated, basified to pH=11 with 25% NaOH (70 ml), and then extracted with ethyl acetate (2×200 ml), dried over sodium sulfate, filtered and concentrated under reduced pressure to obtained biaryl product 46.23 g (83%) as a syrup. HPLC indicated 99.4% product and 0.57% of debrominated staring material.

The above product was dissolved in mixture of ethyl acetate (900 ml) and ethanol (45 ml) and heated at 50° C. 6M aq. HCl (40 ml) was added dropwise over a period often minutes. After 20 minutes, the reaction mixture was cooled to room temperature, and stirring was continued for an additional hour. The resulting white solid was filtered and dried under vacuum at 50° C. for five hours to afford 49.8 grams of the biaryl HCl salt (80%). HPLC indicated pure product.

¹H NMR (DMSO-d₆) δ: 1.92 (m, 4H), 2.52 (m, 2H), 3.12 (m, 2H), 3.82 (m, 1H), 7.51 (m, 2H), 7.75 (m, 1H), 7.82 (br s, 1H), 7.92 (bs d, 2H), 8.12 (brd, 2H), 9.0 (br s, 2H). MH⁺=300, 302 (about 3:1). Pd: 15 ppm.

Method B

A round bottom flask was charged with (4-bromophenyl)(piperidine-4-yl)methanone (20.0 g, 74.6 mmol), 3-chlorophenyl boronic acid (17.4 g, 111 mmol, 1.5 eq), and palladium encapsulated catalyst (Aldrich, Pd EnCat-TPP®, catalyst species PdCl₂(PPh₃)₂) (5.2 g, 0.187 mmol, 0.05 eq). These solids were suspended in isopropanol (570 ml) and allowed to stir for five minutes. To the mixture was added potassium carbonate (30.8 g, 224 mmol, 3 eq) dissolved in H₂O (30 ml). The reaction mixture was heated to 80° C. for 16 hours and deemed complete by LC/MS. The suspension was filtered through a small bed of Celite® and the filtrate was concentrated to dryness. The resulting solids were dissolved in isopropyl acetate (400 ml) and washed with water (3×75 ml). The organic layer was then cooled to 0° C. (ice/water bath) and to this stirring solution was added slowly 6 N HCl until solids crystallized. The solids were filtered and dried in a vacuum oven for 16 hours at 50° C. to afford 16.9 g of compound (68% yield) in >98% HPLC purity. MH⁺=300, 302 (about 3:1). Pd=3 ppm.

Method C

A round bottom flask was charged with (4-bromophenyl)(piperidine-4-yl)methanone (4.00 g, 14.9 mmol), 3-chlorophenyl boronic acid (3.26 g, 20.9 mmol, 1.4 eq), and Fibrecat 1029® (0.70 g, 0.448 mmol, 0.03 eq, Johnson Matthey). These solids were suspended in isopropanol (68 ml) and allowed to stir for five minutes. To this stirring solution was added potassium carbonate (6.18 g, 44.8 mmol, 3 eq.) dissolved in H₂O (12 ml). The resulting solution was heated to 80° C. for 16 hours, at which time the reaction was deemed complete by LC/MS. The reaction mixture was filtered through a small bed of Celite® and the filtrate was concentrated to dryness. The resulting solids were dissolved in isopropyl acetate (100 ml) and washed with water (3×50 ml). The organics were cooled to 0° C., and to this stirring mixture was added slowly 6N HCl until solids crashed out of solution. The solids were filtered and dried for 16 hours at 50° C. in a vacuum oven to afford 2.89 g of compound (72%) in >98% HPLC purity. MH⁺=300, 302 (about 3:1). Pd: 4 ppm

### 6.2. Preparation of (3′-Chloro-biphenyl-4-yl)-(1-pyrimidin-2-yl-piperidine-4-yl)-methanone

A mixture of (3′-chloro-biphenyl-4-yl)-piperidine-4-yl-methanone hydrochloride salt (40 g, 119.4 mmol), 2-chloropyrimidine (19 g, 167.16 mmol, 1.4 eq), potassium carbonate (325 mesh, Aldrich) (49.4 g, 358.2 mmol, 3 eq) and acetonitrile (560 ml) were stirred at 60° C. for 14 hours and deemed complete by LC/MS. The reaction mixture was concentrated, and the residue was dissolved in ethyl acetate (800 ml) and water (200 ml). The aqueous layer was separated and extracted with ethyl acetate (1×200 ml). The organic layers were combined and washed with brine (1×50 ml), dried and concentrated.

### 6.3. Crystallization of (3′-Chloro-biphenyl-4-yl)-(1-pyrimidin-2-yl-piperidine-4-yl)-methanone

The product obtained as described in Example 2 was taken into ethanol (700 ml) and stirred with a mechanical stirrer at 70° C. After 30 minutes, the solid was completely dissolved. At that time, the reaction temperature was decreased to 45° C. and stirred for 4 hours (heterogeneous mixture). The reaction mixture was then stirred at room temperature for 3 hours. The resulting white solid was filtered washed with ethanol (50 ml), dried at 50° C. for 5 hours. This gave the product in 84.4% yield (33.9 g) as a white solid. 100% pure by HPLC.

¹H NMR (CDCl₃) δ: 1.75 (m, 2H), 1.92 (m, 2H), 3.05 (m, 2H), 3.50 (m, 1H), 4.75 (m, 2H), 6.42 (t, 1H), 7.32 (m, 2H), 7.4 (m, 1H), 7.51 (s, 1H), 7.62 (d, 2H), 7.95 (d, 2H), 8.22 (d, 2H). ¹³CNMR (CDCl₃): 28.72, 43.79, 44.34, 110.2, 125.82, 127.78, 127.81, 128.63, 129.39, 130.62, 135.29, 135.49, 142.03, 144.62, 158.13, 161.93, 202.09. MH⁺=378, 380 (about 3:1 ratio). Pd: 2 ppm.

### 6.4. Determination of Particle Size of Particulate Material

Particle size information was obtained using a Sympatec HELOS/KF-MAGIC particle size analyzer equipped with an OASIS dispersion module, RODOS dry disperser, and VIBRI vibratory dry feeder, with a V-shaped chute (Sympatec GmbH, Clausthal-Zellerfeld, Germany). The lens module was: R3; f=100 mm; measuring range=1-175 μm. The air pressure was 1.5 bar and the feed rate was 50%. The data was analyzed using Sympatec's WINDOX 5.1.1.0 software.

### 6.5. Preparation and Bioavailability of Particulate (3′-Chloro-biphenyl-4-yl)-(1-pyrimidin-2-yl-piperidine-4-yl)-methanone

(3′-Chloro-biphenyl-4-yl)-piperidine-4-yl-methanone was micronized using four- and eight-inch Fluid Energy (Hatfield, Pa.) tangential jet mills, with nitrogen gas having a dew point of greater than 40° C.

In one instance, micronization (two passes) yielded a material that had the following characteristics: X₁₀=0.95 μm; X₅₀=3.37 μm; X₉₀=10.66 μm; and all of the particles were smaller than 73.00 μm. When the micronized material was formulated in a liquid suspension, its absolute oral bioavailability in dogs was six times greater than that of the unmicronized compound when administered in a capsule (1.70% vs. 0.27%), and its relative oral bioavailability was 166 times greater (83% vs. 0.50%). Absolute oral bioavailability was determined on the basis of an IV dose, whereas relative oral bioavailability was determined on the basis of an oil-based oral solution.

All patents and patent applications cited above are incorporated herein by reference in their entireties. The full scope of the invention is understood with reference to the appended claims.

What is claimed is:

1. A particulate material, wherein the material is crystalline (3′-chlorobiphenyl-4-yl)(1-(pyrimidin-2-yl)piperidin-4-yl)methanone.

2. The particulate material of claim 1, which has an mean particle size of less than about 5 μm.

3. The particulate material of claim 2, which has an mean particle size of less than about 4 μm.

4. The particulate material of claim 3, which has an mean particle size of less than about 3 μm.

5. The particulate material of claim 1, wherein the crystalline (3′-chlorobiphenyl-4-yl)(1-(pyrimidin-2-yl)piperidin-4-yl)methanone has a X-ray powder diffraction pattern that comprises a peak at about 4.7 degrees 2θ.

6. The particulate material of claim 1, wherein the crystalline (3′-chlorobiphenyl-4-yl)(1-(pyrimidin-2-yl)piperidin-4-yl)methanone has a X-ray powder diffraction pattern that comprises peaks at about 9.3 and 18.8 degrees 2θ.

7. The particulate material of claim 1, wherein the crystalline (3′-chlorobiphenyl-4-yl)(1-(pyrimidin-2-yl)piperidin-4-yl)methanone has a X-ray powder diffraction pattern that comprises peaks at about 19.7 and 22.4 degrees 2θ.

8. The particulate material of claim 1, wherein the crystalline (3′-chlorobiphenyl-4-yl)(1-(pyrimidin-2-yl)piperidin-4-yl)methanone has a X-ray powder diffraction pattern that comprises peaks at about 23.2 and 27.9 degrees 2θ.

9. The particulate material of claim 1, wherein the crystalline (3′-chlorobiphenyl-4-yl)(1-(pyrimidin-2-yl)piperidin-4-yl)methanone has a X-ray powder diffraction pattern that comprises peaks at about 29.6 and 32.2 degrees 2θ.

10. The particulate material of claim 1, wherein the crystalline (3′-chlorobiphenyl-4-yl)(1-(pyrimidin-2-yl)piperidin-4-yl)methanone has a X-ray powder diffraction pattern that comprises peaks at about 32.6 and 37.2 degrees 2θ.

11. The particulate material of claim 1, wherein the crystalline (3′-chlorobiphenyl-4-yl)(1-(pyrimidin-2-yl)piperidin-4-yl)methanone has a X-ray powder diffraction pattern that comprises peaks at about 41.6 and 42.3 degrees 2θ.

12. The particulate material of claim 1, wherein the crystalline (3′-chlorobiphenyl-4-yl)(1-(pyrimidin-2-yl)piperidin-4-yl)methanone has a X-ray powder diffraction pattern that comprises peaks at about 9.3, 27.9 and 42.7 degrees 2θ.

13. The particulate material of claim 1, wherein the crystalline (3′-chlorobiphenyl-4-yl)(1-(pyrimidin-2-yl)piperidin-4-yl)methanone has a X-ray powder diffraction pattern that is substantially the same as that shown in FIG. 1.

14. The particulate material of claim 1, wherein the crystalline (3′-chlorobiphenyl-4-yl)(1-(pyrimidin-2-yl)piperidin-4-yl)methanone has a Raman spectrum that is substantially the same as that shown in FIG. 2.

15. The particulate material of claim 1, wherein the crystalline (3′-chlorobiphenyl-4-yl)(1-(pyrimidin-2-yl)piperidin-4-yl)methanone has a melting point of about 117° C.

16. A pharmaceutical formulation comprising the particulate material of claim 1 and a pharmaceutically acceptable excipient or diluent.

17. The pharmaceutical formulation of claim 16, which is a solid.

18. The pharmaceutical formulation of claim 16, which is a liquid suspension.

19. A method of improving the cognitive performance of a human patient, which comprises administering to the patient an amount of the particulate material of claim 1 sufficient to improve the cognitive performance.

20. A method of treating, managing or preventing a disease or disorder in a patient, which comprises administering to the patient a therapeutically or prophylactically effective amount of the particulate material of claim 1, wherein the disease or disorder is age-associated memory impairment, Alzheimer's disease, Attention-Deficit/Hyperactivity Disorder, autism, Down syndrome, Fragile X syndrome, Huntington's disease, Parkinson's disease, or schizophrenia.
